(19) United States
(12) Patent Application Publication
(10) Pub. No.: US 2026/0124434 A1
(43) Pub. Date: May 7, 2026

(54) GUIDEWIRE

(71) Applicant: FMD CO., LTD., Tokyo (JP)

(72) Inventors: Tsuyoshi TERASHI, Tokyo (JP); Hironori KAWASAKI, Tokyo (JP)

(73) Assignee: FMD CO., LTD., Tokyo (JP)

(21) Appl. No.: 19/376,693

(22) Filed: Oct. 31, 2025

(30) Foreign Application Priority Data
Nov. 5, 2024 (JP) 2024-193509

(51) Int. Cl. A61M 25/09

(52) U.S. Cl. CPC A61M 25/09; A61M 2025/09083; A61M 2025/09133

(57) ABSTRACT

A guidewire includes a core wire, a resin sleeve, a front-end-side coil, a back-end-side coil, and an inside coil. The front-end-side coil includes a front end portion and a back end portion, each of which is fixed to a distal-end-side small-diameter portion of the core wire with solder. The back-end-side coil includes a solder-impregnated back end portion and a solder-unimpregnated front end portion. The solder-impregnated back end portion has an inner space impregnated with solder for fixing the back-end-side coil to the distal-end-side small-diameter portion of the core wire. The solder-unimpregnated front end portion has an inner space that is not impregnated with solder.

CROSS REFERENCES TO RELATED APPLICATIONS

The present invention contains subject matter related to Japanese Patent Application No. 2024-193509 filed in the Japan Patent Office on November 5, 2024, the entire contents of which are incorporated herein by reference.

### BACKGROUND OF THE INVENTION

### 1. Field of the Invention

The present invention relates to a guidewire used to insert a medical apparatus into a living organism.

### 2. Description of the Related Art

A guidewire including a resin sleeve disposed on a distal-end-side small-diameter portion of a core wire has been proposed as a medical guidewire with low resistance and high straight advancing capability (see Japanese Patent No. 4994381).

The guidewire described in Japanese Patent No. 4994381 includes a core wire; a sleeve made of polyimide and disposed on a distal-end-side small-diameter portion of the core wire; and a front-end-side coil and a back-end-side coil disposed on the distal-end-side small-diameter portion of the core wire with the sleeve held therebetween.

The sleeve of the guidewire is capable of rotating around the distal-end-side small-diameter portion of the core wire to facilitate torque transmission (paragraph 0024 of Japanese Patent No. 4994381).

When a guidewire similar to that described in Japanese Patent No. 4994381 is manufactured, a back end portion of the sleeve may melt due to the heat of solder used in the process of fixing (soldering) the back-end-side coil to the distal-end-side small-diameter portion of the core wire.

To avoid this, the back-end-side coil needs to be soldered at a position separated from a back end of the sleeve by a certain distance. Thus, in an actually manufactured guidewire, a front end of the back-end-side coil cannot be disposed in contact with a back end surface of the sleeve. As illustrated in FIG. 12, a gap G (step between the sleeve and the core wire and step between the back-end-side coil and the core wire) is inevitably formed between the sleeve and the back-end-side coil.

When such a gap is formed, the guidewire may receive increased insertion resistance when inserted into a curved blood vessel or the like, and step portions of the gap may come into contact with a vascular plaque, which hinders the insertion of the guidewire. The gap (steps) also degrades the matching properties between the guidewire and a device, such as a catheter, used together with the guidewire.

When the guidewire is bent, for example, by being inserted into a curved blood vessel, the sleeve tends to move slightly in a front-end direction relative to the core wire such that the back end surface of the sleeve moves in the same direction, thereby increasing the gap (distance between the back end surface of the sleeve and the front end of the back-end-side coil) after being bent.

### SUMMARY OF THE INVENTION

The present invention has been made in light of the above-described circumstances, and its object is to provide a guidewire that has no gap between a sleeve and a back-end-side coil, that can be easily inserted into a curved blood vessel and has good matching properties with a device used therewith, and in which a back end portion of the resin sleeve does not melt due to the heat of soldering during manufacture thereof (soldering process of the back-end-side coil).

(1) A guidewire according to the present invention includes a core wire, a resin sleeve, a front-end-side coil, and a back-end-side coil. The resin sleeve is disposed such that the resin sleeve is rotatable around a distal-end-side small-diameter portion of the core wire. The front-end-side coil is formed of a compression coil spring having an outer diameter substantially equal to an outer diameter of the resin sleeve. The front-end-side coil is disposed on a front-end side of the resin sleeve so as to surround the distal-end-side small-diameter portion of the core wire, and has a back end in contact with a front end surface of the resin sleeve. The back-end-side coil is formed of a compression coil spring having an outer diameter substantially equal to the outer diameter of the resin sleeve. The back-end-side coil is disposed on a back-end side of the resin sleeve so as to surround the distal-end-side small-diameter portion of the core wire, and has a front end in contact with a back end surface of the resin sleeve. The front-end-side coil includes a front end portion and a back end portion, each of which is fixed to the distal-end-side small-diameter portion of the core wire with solder. The back-end-side coil includes a solder-impregnated back end portion and a solder-unimpregnated front end portion. The solder-impregnated back end portion has an inner space impregnated with solder for fixing the back-end-side coil to the distal-end-side small-diameter portion of the core wire. The solder-unimpregnated front end portion has an inner space that is not impregnated with solder.

According to the guidewire having the above-described structure, the back-end-side coil, which is formed of the compression coil spring having an outer diameter substantially equal to that of the resin sleeve, has the front end in contact with the back end surface of the resin sleeve. Therefore, no gap (step) is formed between the resin sleeve and the back-end-side coil.

When the guidewire is manufactured (in the soldering process of the back-end-side coil), the back-end-side coil is soldered (solder-impregnated back end portion is formed) at a position separated from the back end of the resin sleeve by a sufficient distance that corresponds to the length of the solder-unimpregnated front end portion. Therefore, the back end portion of the resin sleeve does not melt due to the heat of soldering.

Even when the guidewire is bent, for example, by being inserted into a curved blood vessel, and the resin sleeve moves in the front-end direction relative to the core wire such that the back end surface of the resin sleeve is displaced in the same direction, the solder-unimpregnated front end portion has compression spring characteristics that allow the solder-unimpregnated front end portion to expand. Accordingly, the front end of the back-end-side coil follows the displacement, thereby remaining in contact with the back end surface of the resin sleeve.

When the guidewire is pushed inward while the resin sleeve is caught by a narrowed portion of a blood vessel such that the guidewire is stuck, the resin sleeve tries to move in the back-end direction relative to the core wire such that the back end surface thereof is displaced in the same direction. Even in such a case, since the solder-unimpregnated front end portion has compression spring characteristics, the movement can be stopped, and the compression load applied to the resin sleeve when the guidewire is pushed inward can be reduced. Therefore, kinking or the like of the resin sleeve does not occur.

(2) In the guidewire according to the present invention, preferably, the back-end-side coil has a coil outer diameter (D₄₀) of 0.24 to 0.46 mm, the solder-unimpregnated front end portion has a length (L₄₁) of 0.4 to 1.6 mm, and the solder-impregnated back end portion has a length (L₄₂) of 0.4 to 1.6 mm.

Here, each of the length (L₄₁) of the solder-unimpregnated front end portion and the length (L₄₂) of the solder-impregnated back end portion is a length in the state in which the back-end-side coil is fixed to the distal-end-side small-diameter portion of the core wire.

(3) When the compression coil spring that forms the back-end-side coil includes a portion corresponding to the solder-unimpregnated front end portion and having a free length (length in an uncompressed state) Lₒ and a solid length (length in a fully compressed state) L_{S}, the length (L₄₁) of the solder-unimpregnated front end portion preferably satisfies the following relationship:

(L_{S} + 0.05 mm) ≤ L₄₁≤ (Lₒ - 0.1 mm).

(4) Preferably, the guidewire according to the present invention includes an inside coil formed of a compression coil spring including a front end portion and a back end portion. The front end portion is fixed to the distal-end-side small-diameter portion of the core wire together with the front end portion of the front-end-side coil. The back end portion is fixed to the distal-end-side small-diameter portion of the core wire with solder in an inner space of the resin sleeve.

According to the guidewire having such a structure, the inside coil is disposed in the space defined by an outer peripheral surface of the distal-end-side small-diameter portion of the core wire, an inner peripheral surface of the front-end-side coil, and an inner peripheral surface of the resin sleeve. Thus, the front-end-side coil and the resin sleeve can have the same central axis, so that no step is formed between an outer peripheral surface of the front-end-side coil and an outer peripheral surface of the resin sleeve.

(5) In the guidewire according to the present invention, preferably, the resin sleeve includes a two-layer structure including an inner layer made of polyimide and an outer layer made of polyether block amide.

According to the guidewire having such a structure, since the resin sleeve includes the outer layer made of polyether block amide, the kink resistance of the resin sleeve can be increased, and the adhesion of the resin sleeve to the hydrophilic resin applied to the surface of the resin sleeve can also be increased.

(6) In the guidewire according to the present invention, the back end portion of the front-end-side coil may include a backmost end portion that is a solder-unimpregnated portion having an inner space that is not impregnated with solder.

When the guidewire is pulled outward while the resin sleeve is caught by a narrowed portion of a blood vessel such that the guidewire is stuck, the resin sleeve tries to move in the front-end direction relative to the core wire such that the front end surface thereof is displaced in the same direction. Even in such a case, according to the guidewire having the above-described structure, since the backmost end portion (solder-unimpregnated portion) of the front-end-side coil has compression spring characteristics, the movement can be stopped and the compression load applied to the resin sleeve when the guidewire is pulled outward can be reduced.

(7) In the guidewire according to the present invention, the back end of the front-end-side coil and the front end of the back-end-side coil are closed-ended.

According to the guidewire having such a structure, when the guidewire is rotated (torque is transmitted) while the resin sleeve is caught by a narrowed portion of a blood vessel such that the guidewire is stuck, interference between the back end of the front-end-side coil and the front end surface of the resin sleeve and between the front end of the back-end-side coil and the back end surface of the resin sleeve can be prevented.

According to the guidewire of the present invention, no gap (step) that hinders insertion into a curved blood vessel and that degrades the matching properties with a device used with the guidewire is formed between the resin sleeve and the back-end-side coil.

In addition, when the guidewire is manufactured (in the soldering process of the back-end-side coil), the back end portion of the resin sleeve does not melt due to the heat of soldering.

In addition, even when the back end surface of the resin sleeve is displaced in the front-end direction relative to the core wire, the front end of the back-end-side coil can remain in contact with the back end surface of the resin sleeve.

In addition, the back end surface of the resin sleeve can be prevented from being displaced in the back-end direction relative to the core wire. In addition, the compression load applied to the resin sleeve can be reduced, and kinking or the like of the resin sleeve does not occur.

### BRIEF DESCRIPTION OF THE DRAWINGS

FIG. 1 is a partially broken view of a guidewire according to a first embodiment.

FIG. 2 is an enlarged view of part II in FIG. 1.

FIG. 3 is an enlarged view of part III in FIG. 1.

FIG. 4 is a photograph of a main part of the guidewire according to the first embodiment.

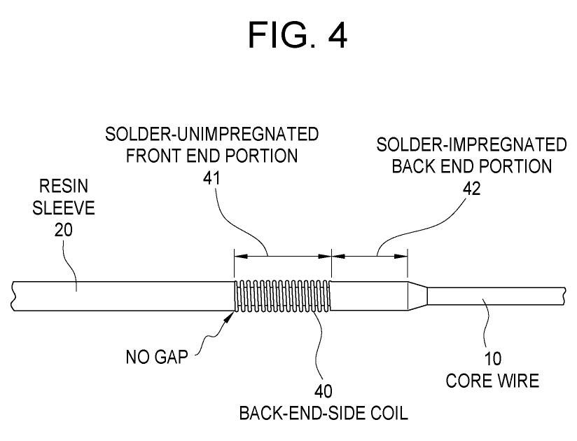

FIGS. 5A and 5B illustrate a resin sleeve included in the guidewire illustrated in FIG. 1.

FIGS. 6A and 6B illustrate a compression coil spring that forms a front-end-side coil of the guidewire illustrated in FIG. 1. FIG. 6A is a side view of the compression coil spring in an uncompressed state, and FIG. 6B is a sectional view of the compression coil spring in an uncompressed state.

FIGS. 7A, 7B, and 7C illustrate a compression coil spring that forms a back-end-side coil of the guidewire illustrated in FIG. 1. FIG. 7A is a side view of the compression coil spring in an uncompressed state, FIG. 7B is a sectional view of the compression coil spring in an

uncompressed state, and FIG. 7C is a sectional view of the compression coil spring in a fully compressed state.

FIGS. 8A and 8B illustrate a compression coil spring that forms an inside coil of the guidewire illustrated in FIG. 1.

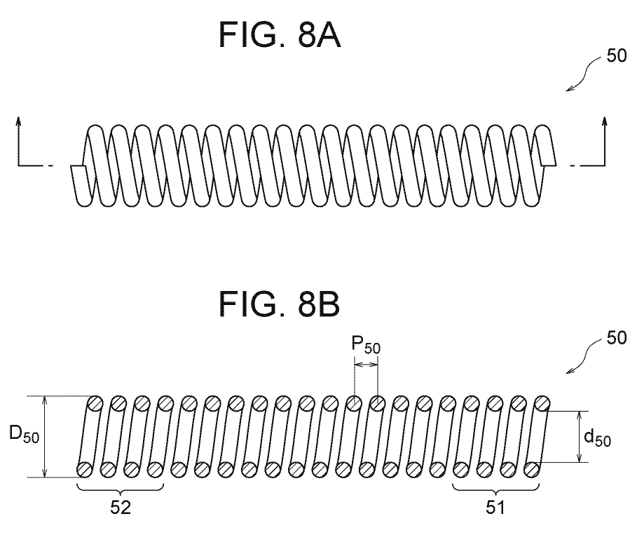

FIG. 9 schematically illustrates the guidewire in a stuck state in which the resin sleeve is caught in a narrowed portion of a blood vessel.

FIG. 10 illustrates a main part of a guidewire according to a second embodiment.

FIG. 11 illustrates a main part of a guidewire according to a third embodiment.

FIG. 12 is a photograph of a guidewire according to the related art in which a gap is formed between a sleeve and a back-end-side coil.

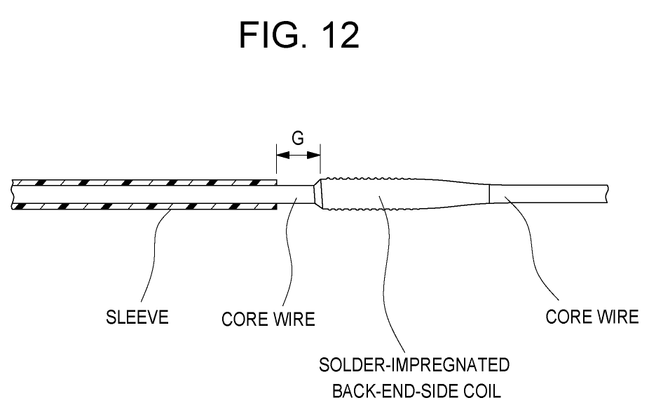

### DESCRIPTION OF THE PREFERRED EMBODIMENTS

### First Embodiment

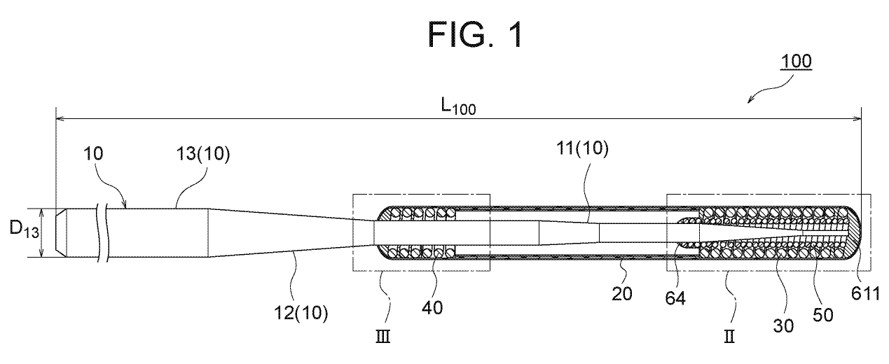

A guidewire 100 according to the present embodiment illustrated in FIGS. 1 to 3 includes a core wire 10, a resin sleeve 20, a front-end-side coil 30, a back-end-side coil 40, and an inside coil 50. The resin sleeve 20 is disposed such that the resin sleeve 20 is rotatable around a distal-end-side small-diameter portion 11 of the core wire 10. The front-end-side coil 30 is formed of a compression coil spring having an outer diameter substantially equal to an outer diameter of the resin sleeve 20. The front-end-side coil 30 is disposed on a front-end side of the resin sleeve 20 so as to surround the distal-end-side small-diameter portion 11 of the core wire 10, and has a back end in contact with a front end surface of the resin sleeve 20. The back-end-side coil 40 is formed of a compression coil spring having an outer diameter substantially equal to the outer diameter of the resin sleeve 20. The back-end-side coil 40 is disposed on a back-end side of the resin sleeve 20 so as to surround the distal-end-side small-diameter portion 11 of the core wire 10, and has a front end in contact with a back end surface of the resin sleeve 20. The inside coil 50 is formed of a compression coil spring including a front end portion 51 and a back end portion 52. The front end portion 51 is fixed to the distal-end-side small-diameter portion 11 of the core wire 10 together with a front end portion 31 of the front-end-side coil 30 with solder 61. The back end portion 52 is fixed to the distal-end-side small-diameter portion 11 of the core wire 10 with solder 64 in an inner space of the resin sleeve 20. The front-end-side coil 30 includes the front end portion 31 and a back end portion 32, each of which is fixed to the distal-end-side small-diameter portion 11 of the core wire 10 with solder 61, 62. The back-end-side coil 40 includes a solder- impregnated back end portion 42 and a solder-unimpregnated front end portion 41. The solder-impregnated back end portion 42 has an inner space impregnated with solder 63 for fixing the back-end-side coil 40 to the distal-end-side small-diameter portion 11 of the core wire 10. The solder-unimpregnated front end portion 41 has an inner space that is not impregnated with solder (not soldered).

The guidewire 100 of the present embodiment includes the core wire 10, the resin sleeve 20, the front-end-side coil 30, the back-end-side coil 40, and the inside coil 50.

The core wire 10 of the guidewire 100 includes a proximal-end-side large-diameter portion 13, a tapered portion 12 whose diameter decreases in a front-end direction (distal direction), and a distal-end-side small-diameter portion 11 whose diameter decreases stepwise in the front-end direction.

The distal-end-side small-diameter portion 11, the tapered portion 12, and the proximal-end-side large-diameter portion 13 are formed integrally from the same linear material (for example, a cylindrical rod member).

The material of the core wire 10 is not particularly limited, and examples of the material include stainless steel, such as SUS316 and SUS304; gold, platinum, aluminum, tungsten, tantalum, and alloys thereof; and nickel-titanium alloy and other metals. In this embodiment, the core wire 10 is made of stainless steel.

A water-repellent resin layer (not illustrated) is formed on an outer peripheral surface of the core wire 10.

The water-repellent resin layer may be made of any resin that is used for medical purposes and that has water repellency. Preferred examples of such a resin include fluorine-based resins, such as PTFE.

The guidewire 100 preferably has an overall length (L₁₀₀) of 1300 to 3500 mm. A preferred example of the overall length (L₁₀₀) is 2015 mm.

The proximal-end-side large-diameter portion 13 preferably has an outer diameter (D₁₃) of 0.25 to 0.46 mm. A preferred example of the outer diameter (D₁₃) is 0.35 mm.

A maximum outer diameter of the distal-end-side small-diameter portion 11 is not particularly limited as long as the maximum outer diameter is less than an inner diameter of the resin sleeve 20. A preferred example of the maximum outer diameter is 0.190 mm.

The resin sleeve 20 of the guidewire 100 is disposed such that the resin sleeve 20 is rotatable around the distal-end-side small-diameter portion 11 of the core wire 10.

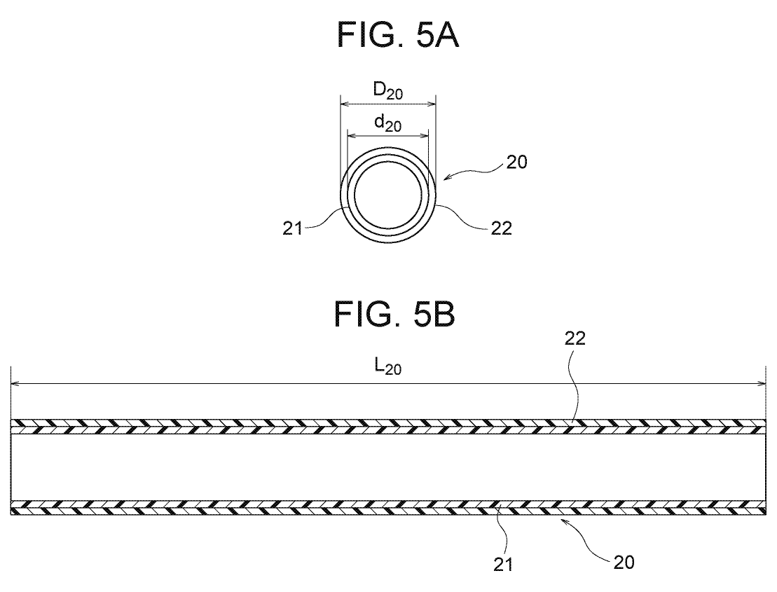

As illustrated in FIGS. 5A and 5B, the resin sleeve 20 includes a two-layer structure including an inner layer 21 made of polyimide and an outer layer 22 made of polyether block amide (PEBAX®).

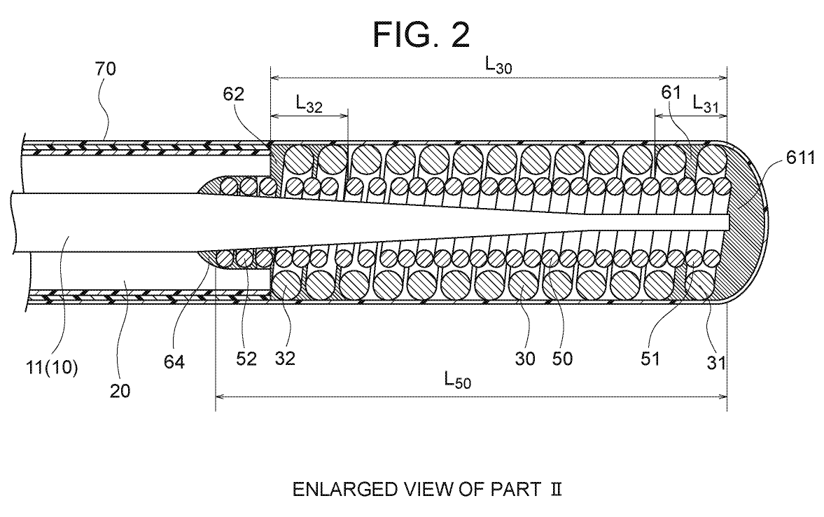

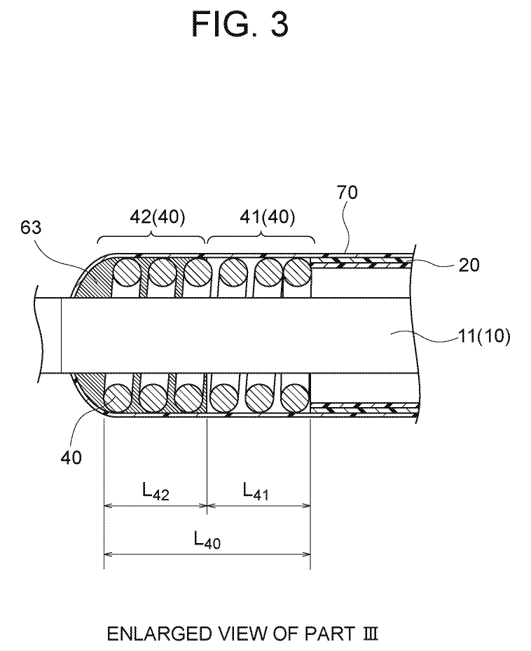

As illustrated in FIGS. 2 and 3, a hydrophilic resin layer 70 is formed on a surface of the resin sleeve 20.

Since the resin sleeve 20 includes the outer layer 22 made of polyether block amide, the resin sleeve 20 has a higher kink resistance and higher adhesion to the hydrophilic resin layer 70 on the surface of the resin sleeve 20 (outer layer 22) than a sleeve according to the related art formed only of polyimide.

The resin sleeve 20 preferably has a length (L₂₀) of 100 to 500 mm. A preferred example of the length (L₂₀) is 330 mm.

The resin sleeve 20 preferably has an outer diameter (D₂₀) of 0.200 to 0.460 mm. A preferred example of the outer diameter (D₂₀) is 0.315 mm.

The resin sleeve 20 preferably has an inner diameter (d₂₀) of 0.100 to 0.360 mm. A preferred example of the inner diameter (d₂₀) is 0.215 mm.

The ratio between the thickness of the inner layer 21 and the thickness of the outer layer 22 is preferably 4:6 to 6:4. In the preferred example illustrated in FIGS. 5A and 5B, the ratio is 5:5.

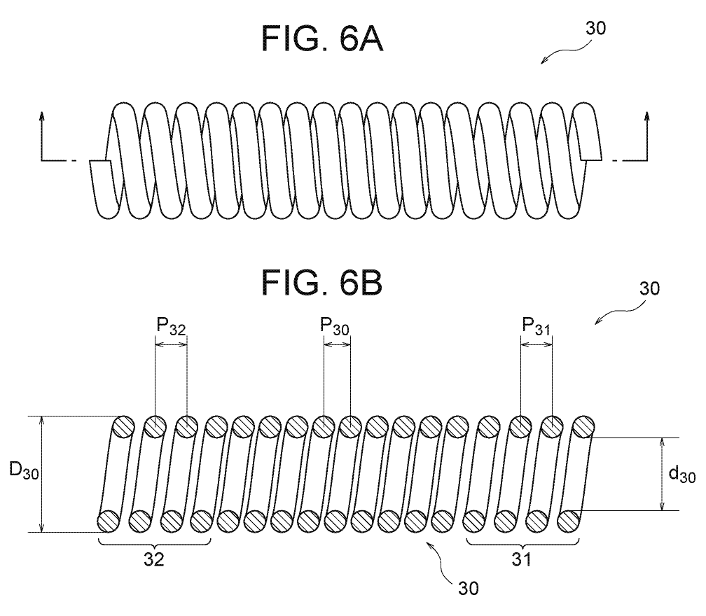

The front-end-side coil 30 of the guidewire 100 is formed of a compression coil spring illustrated in FIGS. 6A and 6B, and is disposed on the front-end side of the resin sleeve 20 so as to surround the distal-end-side small-diameter portion 11 of the core wire 10. The front-end-side coil 30 has a back end in contact with the front end surface of the resin sleeve 20.

The front-end-side coil 30 is fixed to the distal-end-side small-diameter portion 11 of the core wire 10 with the solder 61 in the inner space of the front end portion 31 and the solder 62 in the inner space of the back end portion 32. In FIGS. 1 and 2, the solder 61 forms a front-end tip 611. The solder 61 and the solder 62 in the inner space of the front end portion 31 and the inner space of the back end portion 32 may be, for example, Au-Sn-based solder or Ag-Sn-based solder.

The front-end-side coil 30 has an outer diameter substantially equal to an outer diameter of the resin sleeve 20. More specifically, when the outer diameter of the front-end-side coil 30 is D₃₀, the value of (D₃₀/D₂₀) may be 0.9 to 1.2, preferably 0.9 to 1.1.

A preferred example of the outer diameter (D₃₀) of the front-end-side coil 30 is 0.34 mm.

A preferred example of an inner diameter (d₃₀) of the front-end-side coil 30 is 0.22 mm.

The front-end-side coil 30 preferably has a wire diameter of 0.050 to 0.080 mm. A preferred example of the wire diameter is 0.060 mm.

The front-end-side coil 30 preferably has a length [length (L₃₀ in FIG. 2) when the front-end-side coil 30 is fixed to the distal-end-side small-diameter portion 11 of the core wire 10] of 20 to 80 mm. A preferred example of the length (L₃₀) is 50 mm.

The front end portion 31 having the inner space impregnated with the solder 61 preferably has a length (L₃₁) of 0.3 to 1.0 mm. A preferred example of the length (L₃₁) is 0.6 mm.

The back end portion 32 having the inner space impregnated with the solder 62 preferably has a length (L₃₂) of 0.4 to 1.2 mm. A preferred example of the length (L₃₂) is 0.8 mm.

As illustrated in FIGS. 6A and 6B, the compression coil spring that forms the front-end-side coil 30 is formed such that a coil pitch (p₃₁) in a section corresponding to the front end portion 31 and a coil pitch (p₃₂) in a section corresponding to the back end portion 32 are greater than a coil pitch (p₃₀) in a middle section.

Thus, in a soldering process for fixing the front-end-side coil 30 (front end portion 31 and back end portion 32) to the distal-end-side small-diameter portion 11 of the core wire 10, the inner spaces of the front end portion 31 and the back end portion 32 can be easily impregnated with solder.

The compression coil spring that forms the front-end-side coil 30 preferably has a free length of 20 to 80 mm. A preferred example of the free length is 52 mm.

The coil pitch (p₃₀) of the compression coil spring in the middle section is preferably 0.05 to 0.12 mm. A preferred example of the coil pitch (p₃₀) is 0.08 mm.

The coil pitch (p₃₁) in the section corresponding to the front end portion 31 and the coil pitch (p₃₂) in the section corresponding to the back end portion 32 are preferably 0.07 to 0.15 mm. A preferred example of the coil pitch (p₃₁) and the coil pitch (p₃₂) is 0.095 mm.

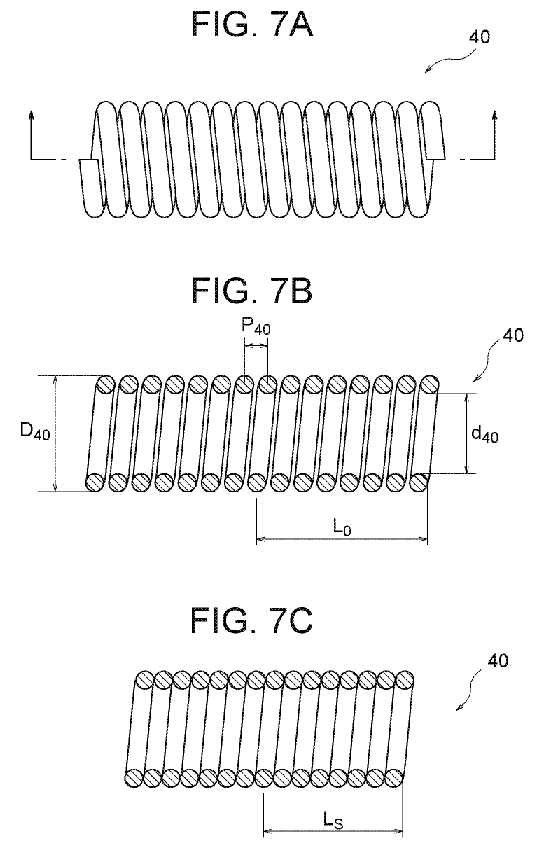

The back-end-side coil 40 of the guidewire 100 is formed of a compression coil spring illustrated in FIGS. 7A to 7C, and is disposed on the back-end side of the resin sleeve 20 so as to surround the distal-end-side small-diameter portion 11 of the core wire 10. The back-end-side coil 40 has a front end in contact with the back end surface (pressing the back end surface) of the resin sleeve 20.

As illustrated in FIG. 3, the back-end-side coil 40 includes the solder-impregnated back end portion 42 and the solder-unimpregnated front end portion 41. The solder-impregnated back end portion 42 has the inner space impregnated with the solder 63 for fixing the back-end-side coil 40 to the distal-end-side small-diameter portion 11 of the core wire. The solder-unimpregnated front end portion 41 has the inner space that is not impregnated with solder (not soldered). Thus, the back-end-side coil 40 is fixed to the distal-end-side small-diameter portion 11 of the core wire 10 with the solder 63 that is present only in the inner space of the solder-impregnated back end portion 42. The solder 63 in the inner space of the solder-impregnated back end portion 42 may be, for example, Au-Sn-based solder or Ag-Sn-based solder.

The solder-unimpregnated front end portion 41 having the inner space that is not impregnated with solder has compression spring characteristics of the compression coil spring.

The back-end-side coil 40 has an outer diameter substantially equal to the outer diameter of the resin sleeve 20. More specifically, when the outer diameter of the back-end-side coil 40 is D₄₀, the value of (D₄₀/D₂₀) may be 0.9 to 1.2, preferably 0.9 to 1.1.

The back-end-side coil 40 preferably has an outer diameter (D₄₀) of 0.24 to 0.46 mm, more preferably 0.32 to 0.36 mm. A preferred example of the outer diameter (D₄₀) is 0.34 mm.

The back-end-side coil 40 preferably has an inner diameter (d₄₀ ) of 0.08 to 0.40 mm, more preferably 0.22 to 0.26 mm. A preferred example of the inner diameter (d₄₀) is 0.24 mm.

The back-end-side coil 40 preferably has a wire diameter of 0.03 to 0.08 mm. A preferred example of the wire diameter is 0.050 mm.

The back-end-side coil 40 preferably has a length [length (L₄₀in FIG. 3) when the back-end-side coil 40 is fixed to the distal-end-side small-diameter portion 11 of the core wire 10] of 1.0 to 3.0 mm. A preferred example of the length (L₄₀) is 1.70 mm.

The ratio between the length (L₄₁) of the solder-unimpregnated front end portion 41 and the length (L₄₂) of the solder-impregnated back end portion 42 is preferably 4:6 to 6:4. In the preferred example illustrated in FIG. 3, the ratio is 5:5.

The length (L₄₁) of the solder-unimpregnated front end portion 41 is preferably 0.4 to 1.6 mm. A preferred example of the length (L₄₁) is 0.85 mm.

When the length (L₄₁) of the solder-unimpregnated front end portion 41 is 0.4 mm or more, the resin sleeve 20 and the solder-impregnated back end portion 42 are separated by a sufficient distance. Accordingly, the back end portion of the resin sleeve 20 can be reliably prevented from receiving the heat of soldering (formation of the solder-impregnated back end portion) during manufacture of the guidewire 100 (in the soldering process of the back-end-side coil 40). In addition, the solder-unimpregnated front end portion 41 can exhibit sufficient compression spring characteristics.

When the length (L₄₁) of the solder-unimpregnated front end portion 41 is 1.6 mm or less, the back-end-side coil 40 including the solder-unimpregnated front end portion 41 can be reliably fixed to the distal-end-side small-diameter portion 11 of the core wire 10 by the solder-impregnated back end portion 42.

The length (L₄₂) of the solder-impregnated back end portion 42 is preferably 0.4 to 1.6 mm. A preferred example of the length (L₄₂) is 0.85 mm.

When the length (L₄₂) of the solder-impregnated back end portion 42 is 0.4 mm or more, the back-end-side coil 40 including the solder-impregnated back end portion 42 can be reliably fixed to the distal-end-side small-diameter portion 11 of the core wire 10 by the solder-impregnated back end portion 42.

When the length (L₄₂) of the solder-impregnated back end portion is 1.6 mm or less, the back end portion of the resin sleeve 20 can be reliably prevented from receiving the heat of soldering (formation of the solder-impregnated back end portion) during manufacture of the guidewire 100 (in the soldering process of the back-end-side coil 40). In addition, the solder-unimpregnated front end portion 41 can exhibit sufficient compression spring characteristics and sufficient flexibility.

FIGS. 7A, 7B, and 7C illustrate the compression coil spring that forms the back-end-side coil 40. FIG. 7A is a side view of the compression coil spring in an uncompressed state. FIG. 7B is a sectional view of the compression coil spring in the uncompressed state. FIG. 7C is a sectional view of the compression coil spring in a fully compressed state.

The compression coil spring illustrated in FIGS. 7A to 7C preferably has a free length (length in the uncompressed state illustrated in FIGS. 7A and 7B) of 1.4 to 3.5 mm. A preferred example of the free length is 2.0 mm.

A portion corresponding to the solder-unimpregnated front end portion 41 preferably has a free length (Lₒ) of 0.7 to 1.75 mm. A preferred example of the free length (Lₒ) is 1.0 mm.

The compression coil spring illustrated in FIGS. 7A to 7C preferably has a solid length (length in the fully compressed state illustrated in FIG. 7C) of 0.8 to 2.6 mm. A preferred example of the solid length is 1.42 mm.

The portion corresponding to the solder-unimpregnated front end portion 41 preferably has a solid length (L_{S}) of 0.4 to 1.3 mm. A preferred example of the solid length (L_{S}) is 0.71 mm.

The compression coil spring illustrated in FIG. 7B preferably has a coil pitch (p₄₀) of 0.06 to 0.08 mm. A preferred example of the coil pitch (p₄₀) is 0.07 mm.

When the compression coil spring that forms the back-end-side coil 40 includes a portion corresponding to the solder-unimpregnated front end portion 41 and having the free length Lₒ and the solid length L_{S}, the length (L₄₁) of the solder-unimpregnated front end portion 41 preferably satisfies the following relationship: (L_{S} + 0.05 mm) ≤ L₄₁ ≤ (Lₒ - 0.1 mm).

When the length (L₄₁) of the solder-unimpregnated front end portion 41 is (Lₒ - 0.1 mm) or less, the compression coil spring (portion thereof) forming the solder-unimpregnated front end portion 41 is capable of expanding by at least 0.1 mm.

When the guidewire 100 is bent, for example, by being inserted into a curved blood vessel, the resin sleeve 20 moves in the front-end direction relative to the core wire 10, and the back end surface thereof is displaced in the same direction. However, the displacement of the back end surface of the resin sleeve 20 in the front-end direction is normally less than or equal to 0.1 mm, and the front end of the back-end-side coil 40 can sufficiently follow the displacement and reliably remain in contact with the back end surface of the resin sleeve 20.

When the length (L₄₁) of the solder-unimpregnated front end portion 41 is (L_{S} + 0.05 mm) or more, the compression coil spring (portion thereof) forming the solder-unimpregnated front end portion 41 is capable of being compressed by at least 0.05 mm.

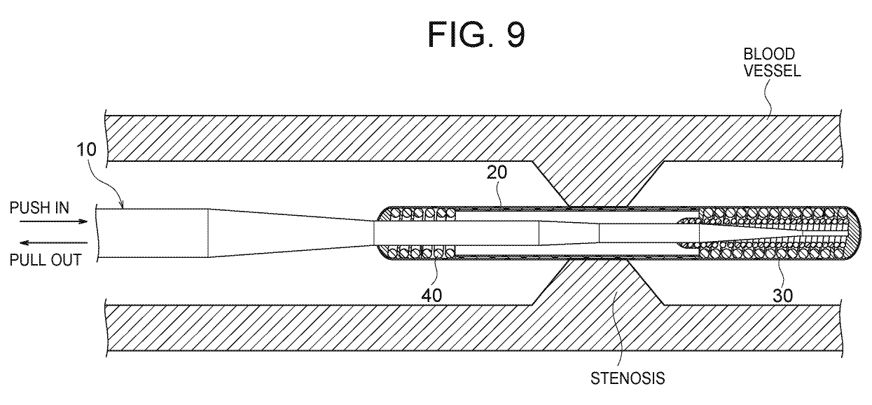

Referring to FIG. 9, when, for example, the guidewire 100 is pushed inward (PUSH IN) while the resin sleeve 20 is caught by a narrowed portion of a blood vessel such that the guidewire 100 is stuck, the resin sleeve 20 tries to move in a back-end direction relative to the core wire 10 such that the back end surface thereof is displaced in the same direction. Even in such a case, since the solder-unimpregnated front end portion 41 has compression spring characteristics, the movement can be stopped and the compression load applied to the resin sleeve 20 when the guidewire 100 is pushed inward can be sufficiently reduced.

The inside coil 50 of the guidewire 100 is formed of a compression coil spring including the front end portion 51 and the back end portion 52. The front end portion 51 is fixed to the distal-end-side small-diameter portion 11 of the core wire 10 together with the front end portion 31 of the front-end-side coil 30 (that is, with the solder 61). The back end portion 52 is fixed to the distal-end-side small-diameter portion 11 of the core wire 10 with the solder 64 in the inner space of the resin sleeve 20. The inside coil 50 is disposed in a space defined by an outer peripheral surface of the distal-end-side small-diameter portion 11 of the core wire 10, an inner peripheral surface of the front-end-side coil 30, and an inner peripheral surface of the resin sleeve 20.

The solder 64 may be, for example, Au-Sn-based solder or Ag-Sn-based solder.

Since the inside coil 50 is disposed in the above-described space, the front-end-side coil 30 and the resin sleeve 20 can have the same central axis, so that no step is formed between an outer peripheral surface of the front-end-side coil 30 and an outer peripheral surface of the resin sleeve 20.

The inside coil 50 preferably has an outer diameter (D₅₀) slightly less than the inner diameter (d₃₀) of the front-end-side coil 30. A preferred example of the outer diameter (D₅₀) is 0.19 mm.

The inner diameter (d₅₀) of the inside coil 50 is greater than an outer diameter of the distal-end-side small-diameter portion 11 around which the inside coil 50 is disposed. A preferred example of the inner diameter (d₅₀) is 0.12 mm.

The inside coil 50 preferably has a length [length (L₅₀ in FIG. 2) when the inside coil 50 is fixed to the distal-end-side small-diameter portion 11 of the core wire 10] of 60 to 70 mm. A preferred example of the length (L₅₀) is 65 mm.

The inside coil 50 preferably has a wire diameter of 0.02 to 0.05 mm. A preferred example of the wire diameter is 0.035 mm.

The compression coil spring that forms the inside coil 50 preferably has a free length of 30 to 120 mm. A preferred example of the free length is 70 mm.

The compression coil spring preferably has a coil pitch (p₅₀) of 0.025 to 0.08 mm. A preferred example of the coil pitch (p₅₀) is 0.055 mm.

According to the guidewire 100 of the present embodiment, the back-end-side coil 40 formed of a compression coil spring has the front end in contact with the back end surface (pressing the back end surface) of the resin sleeve 20. Therefore, no gap that hinders insertion into the curved blood vessel and that degrades the matching properties with a device used with the guidewire 100 is formed between the resin sleeve 20 and the back-end-side coil 40.

In addition, since the compression coil spring that forms the back-end-side coil 40 has an outer diameter substantially equal to that of the resin sleeve 20, no step is formed between the resin sleeve 20 and the back-end-side coil 40.

When the guidewire 100 is manufactured (in the soldering process of the back-end-side coil 40), the back-end-side coil 40 is soldered (solder-impregnated back end portion 42 is formed) at a position separated from the back end of the resin sleeve 20 by a sufficient distance that corresponds to the length (L₄₁) of the solder-unimpregnated front end portion 41. Therefore, the back end portion of the resin sleeve 20 does not melt due to the heat of soldering.

Even when the guidewire 100 is bent, for example, by being inserted into a curved blood vessel, and the resin sleeve 20 moves in the front-end direction relative to the core wire 10 such that the back end surface of the resin sleeve 20 is displaced in the same direction, the solder-unimpregnated front end portion 41 has compression spring characteristics that allow the solder-unimpregnated front end portion 41 to expand. Accordingly, the front end of the back-end-side coil 40 follows the displacement, thereby remaining in contact with the back end surface of the resin sleeve 20.

Referring to FIG. 9, when the guidewire 100 is pushed inward (PUSH IN) while the resin sleeve 20 is caught by a narrowed portion of a blood vessel such that the guidewire 100 is stuck, the resin sleeve 20 tries to move in the back-end direction relative to the core wire 10 such that the back end surface thereof is displaced in the same direction. Even in such a case, since the solder-unimpregnated front end portion 41 has compression spring characteristics, the movement can be stopped, and the compression load applied to the resin sleeve 20 when the guidewire is pushed inward can be reduced. Therefore, kinking or the like of the resin sleeve 20 does not occur.

### Second Embodiment

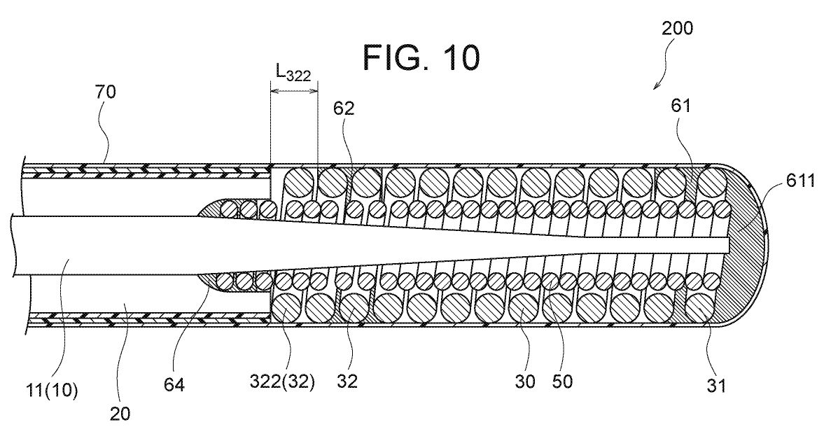

FIG. 10 illustrates a main part of a guidewire 200 according to this embodiment. The guidewire 200 is similar to the guidewire of the first embodiment except that the back end portion 32 of the front-end-side coil 30 includes a backmost end portion 322 having an inner space that is not impregnated with the solder 62.

Referring to FIG. 9, when the guidewire 200 is pulled outward (PULL OUT) while the resin sleeve 20 is caught by a narrowed portion of a blood vessel such that the guidewire 200 is stuck, the resin sleeve 20 tries to move in the front-end direction relative to the core wire 10 such that the front end surface thereof is displaced in the same direction. Even in such a case, according to the guidewire 200 of the present embodiment, since the backmost end portion 322 (solder-unimpregnated portion) of the front-end-side coil 30 has compression spring characteristics, the movement can be stopped, and the compression load applied to the resin sleeve 20 when the guidewire 200 is pulled outward can be reduced.

The backmost end portion 322 having the inner space that is not impregnated with the solder 62 preferably has a length (L₃₂₂) of 0.5 to 1.5 mm. A preferred example of the length (L₃₂₂) is 0.85 mm.

### Third Embodiment

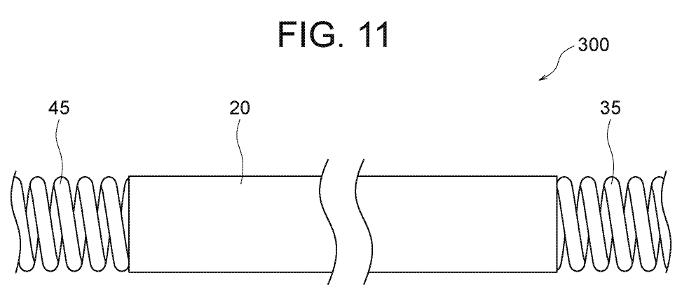

FIG. 11 illustrates a main part of a guidewire 300 according to this embodiment. The guidewire 300 is similar to the guidewire of the first embodiment except that each of a back end of a front-end-side coil 35 in contact with the front end surface of the resin sleeve 20 and a front end of a back-end-side coil 45 in contact with the back end surface of the resin sleeve 20 is closed-ended.

According to the guidewire 300 of the present embodiment, when the guidewire 300 is rotated (torque is transmitted) while the resin sleeve 20 is caught by a narrowed portion of a blood vessel such that the guidewire 300 is stuck as illustrated in FIG. 9, interference between the back end of the front-end-side coil 35 and the front end surface of the resin sleeve 20 and between the front end of the back-end-side coil 45 and the back end surface of the resin sleeve 20 can be prevented.

What is claimed is:

1. A guidewire comprising:
a core wire;
a resin sleeve disposed such that the resin sleeve is rotatable around a distal-end-side small-diameter portion of the core wire;
a front-end-side coil formed of a compression coil spring having an outer diameter substantially equal to an outer diameter of the resin sleeve, the front-end-side coil being disposed on a front-end side of the resin sleeve so as to surround the distal-end-side small-diameter portion of the core wire and having a back end in contact with a front end surface of the resin sleeve; and
a back-end-side coil formed of a compression coil spring having an outer diameter substantially equal to the outer diameter of the resin sleeve, the back-end-side coil being disposed on a back-end side of the resin sleeve so as to surround the distal-end-side small-diameter portion of the core wire and having a front end in contact with a back end surface of the resin sleeve,
wherein the front-end-side coil includes a front end portion and a back end portion, each of which is fixed to the distal-end-side small-diameter portion of the core wire with solder, and
wherein the back-end-side coil includes a solder-impregnated back end portion and a solder-unimpregnated front end portion, the solder-impregnated back end portion having an inner space impregnated with solder for fixing the back-end-side coil to the distal-end-side small-diameter portion of the core wire, the solder-unimpregnated front end portion having an inner space that is not impregnated with solder.

2. The guidewire according to claim 1, wherein the back-end-side coil has a coil outer diameter of 0.24 to 0.46 mm, the solder-unimpregnated front end portion has a length of 0.4 to 1.6 mm, and the solder-impregnated back end portion has a length of 0.4 to 1.6 mm.

3. The guidewire according to claim 2, wherein when the compression coil spring that forms the back-end-side coil includes a portion corresponding to the solder-unimpregnated front end portion and having a free length Lₒ and a solid length L_{S} the length L₄₁of the solder-unimpregnated front end portion satisfies the following relationship:
(L_{S}+ 0.05 mm) ≤ L₄₁≤ (Lₒ - 0.1 mm).

4. The guidewire according to claim 1, comprising:
an inside coil formed of a compression coil spring including
a front end portion fixed to the distal-end-side small-diameter portion of the core wire together with the front end portion of the front-end-side coil, and
a back end portion fixed to the distal-end-side small-diameter portion of the core wire with solder in an inner space of the resin sleeve.

5. The guidewire according to claim 1, wherein the resin sleeve includes a two-layer structure including an inner layer made of polyimide and an outer layer made of polyether block amide.

6. The guidewire according to claim 1, wherein the back end portion of the front-end-side coil includes a backmost end portion that is a solder-unimpregnated portion having an inner space that is not impregnated with solder.

7. The guidewire according to claim 1, wherein the back end of the front-end-side coil and the front end of the back-end-side coil are closed-ended.
